(19) United States
(12) Patent Application Publication
(10) Pub. No.: US 2011/0238092 A1
(43) Pub. Date: Sep. 29, 2011

(54) ULTRAHIGH MOLECULAR WEIGHT POLYETHYLENE YARN

(75) Inventors: Claudia Maria Vaz, Maastricht (NL); Erik Becker, Maastricht (NL); Harm van der Werff, Bunde (NL)

(73) Assignee: DSM IP ASSETS B.V., Heerlen (NL)

(21) Appl. No.: 13/000,131

(22) PCT Filed: Jun. 18, 2009
(86) PCT No.: PCT/EP2009/057607

§ 371 Date: Jun. 13, 2011

(30) Foreign Application Priority Data
Jun. 20, 2008 (EP) 08158697.6

(51) Int. Cl. D01F 6/04; D01F 6/30; D03D 9/00; A61B 17/04; A61B 17/00; D01D 5/12

(52) U.S. Cl. 606/151; 428/401; 428/400; 442/1; 606/228; 264/210.8

(57) ABSTRACT

The invention relates to an ultrahigh molecular weight polyethylene (UHMwPE) yarn and to a method for producing thereof. The invention also relates to a medical device comprising the yarn. A UHMwPE yarn is provided having a diameter of 30 μm or more and a residual spin solvent of less than 100 ppm, wherein the yarn is a gel-spun monofilament.

### TECHNICAL FIELD OF THE INVENTION

The invention relates to an ultrahigh molecular weight polyethylene (UHMwPE) yarn and to a method for producing thereof. The invention also relates to a medical device comprising the yarns.

### BACKGROUND OF THE INVENTION

UHMwPE yarn has been described in various publications, including EP 0205960 A, EP 0213208A1, U.S. Pat. No. 4,413,110, WO 01/73173 A1, and Advanced Fiber Spinning Technology, Ed. T. Nakajima, Woodhead Publ. Ltd (1994), ISBN 1-855-73182-7, and references cited therein. In these publications, UHMwPE multifilaments yarns are made by a gel spinning process. Gel spun UHMwPE multifilament yarns have favorable mechanical properties, like a high modulus and a high tensile strength. The gel spun filaments which form the yarns however have the drawback that they contain residual spin solvent. Although the level of residual spin solvent normally is low, it is typically still too high to make the filament suitable for use in medical applications like sutures and cables, since the residual spin solvent may cause unwanted reactions of the human or animal body, like for instance inflammation. Residual spin solvent thus needs to eliminated, e.g. by a cleaning process which may relate to evaporation, extraction or a combination of evaporation and extraction. Such a cleaning process is rather cumbersome, and typically the mechanical properties of the yarns deteriorate during the cleaning process. Furthermore the smoothness of the surface of the filament may be adversely affected during the cleaning process, so that the coefficient of friction of the filament increases. This makes it for example more difficult to stitch a wound with a suture comprising the filament. Also, the rate at which the yarn creeps will increase.

Multifilament yarns have another drawback, in that the small void areas or interstitial spaces between the filaments of multifilament yarns may act as harbor for infectious matters such as bacteria.

EP0740002A1 discloses a yarn made by fusing gel spun polyolefin filaments having a smaller diameter. A yarn is made by this process having a sufficient diameter for fishing lines, with some characteristics of a monofilament. However, this process may potentially lead to internal pores or open pores which may harbor bacteria similar to multifilament yarns. Also, the fused multifilaments show a relatively high surface roughness, making them less suitable for medical applications.

### OBJECT OF THE INVENTION

It is an object of the present invention to provide an improved UHMwPE yarn. The improvement may for example be to overcome one or more of the above limitations.

### DISCLOSURE OF THE INVENTION

Therefore, a UHMwPE yarn is provided having a diameter of 30 μm or more and a spin solvent residue of less than 100 ppm, wherein the yarn is a monofilament.

The monofilament which forms the present yarn has a diameter large enough for use as a yarn in medical applications, e.g. as a suture, from handling perspective and mechanical properties. The monofilament thus does not need to be twisted to make a yarn as in multifilaments, hence reducing the required number of steps and providing a simplified method of making a yarn. Furthermore, the closed structure of the monofilament has no space for attracting bacteria.

For the purposes of the present invention, a yarn is herein understood to mean a product or an article the length dimension of which is much greater than its transverse diameter that can be used as an end-product or for making various other articles. Therefore a yarn herein includes both a yarn made of a plurality of monofilaments and a yarn made of a single monofilament.

A monofilament is herein understood to mean a filament obtainable from a single spin hole. It is noted that a monofilament herein does not include a fused multifilament yarn having some monofilament characteristics, such as the one described in EP0740002A1. For the purposes of the present invention, a monofilament is an elongated body the length dimension of which is much greater than its transverse diameter. Preferably, the monofilaments have a substantially circular or elliptical cross-section. In comparison to the yarn which is a monofilament, a multifilament yarn is herein understood as an elongated body comprising a plurality of individual monofilaments which are arranged to make up a single yarn. The multifilament yarn may potentially include partially fused filaments.

UHMwPE is herein defined as a polyethylene having an intrinsic viscosity (IV) of more than 5 dl/g. Intrinsic viscosity is a measure for molar mass (also called molecular weight) that can more easily be determined than actual molar mass parameters such as Mn and Mw. The IV is determined according to method PTC-179 (Hercules Inc. Rev. Apr. 29, 1982) at 135° C. in decalin, the dissolution time being 16 hours, with DBPC as the anti-oxidant in an amount of 2 g/l solution, and the viscosity at different concentrations is extrapolated to zero concentration. Because of their long molecule chains, stretched polyolefin fibers with an IV of more than 5 dl/g have very good mechanical properties, such as a high tensile strength, modulus, and energy absorption at break. More preferably, a polyethylene with an IV of more than 10 dl/g is chosen. This is because such gel-spun UHMwPE yarn offers a combination of high strength, low relative density, good hydrolysis resistance, and excellent wear properties, making it particularly suited for use in various biomedical applications, including implants.

Preferably, the UHMwPE of the present invention is a linear polyethylene, i.e. a polyethylene with less than one side chain or branch per 100 carbon atoms, and preferably less than one side chain per 300 carbon atoms, a branch generally containing at least 10 carbon atoms. Preferably, only polyethylene is present, but alternatively the polyethylene may further contain up to 5 mol % of alkenes that may or may not be copolymerized with it, such as propylene, butene, pentene, 4-methylpentene or octene. The polyethylene may further contain additives that are customary for such fibres, such as anti-oxidants, thermal stabilizers, colorants, etc., up to 15 weight %, preferably 1-10 weight %. The UHMwPE may further be added with a polyethylene with lower molecular weight, preferably up to 10 mol %.

The diameter of a monofilament is herein understood to mean the average diameter D of the yarn calculated from the dtex (g/10 km) of the yarn according to formula 1:

D(μm)=(4/π·ρ⁻¹·dtex·10⁻⁷)^{1/2}·10⁶ Formula 1

wherein density ρ of the monofilament is assumed to be 970 kg/m³.

The yarn according to the present invention has a diameter which is large enough to be used as a suture. Filaments having a high diameter are more robust during handling (for example with regard to friction) by a surgeon and more abrasion resistant. A yarn having a diameter of 30 μm can be used as a suture of USP 9-0. In another embodiment, the yarn has a diameter of 40 μm, which can be used as a suture of USP 8-0. A yarn having a diameter of 50 μm may be used as a suture of USP 7-0. The higher diameter provides a higher total strength, although typically the specific strength decreases with a diameter increase. The diameter of the yarn is preferably not higher than 150 μm, since it is difficult to eliminate the residual solvent to the level of 100 ppm or less. Even more preferably, the diameter of the yarn is not higher than 100 μm.

The residual spin solvent is herein understood to mean the content of the solvent used in making the monofilament, which is still remaining in the final monofilament. In the process of making the yarn, any of the known solvents for gel spinning of UHMwPE can be used. Suitable examples of spinning solvents include aliphatic and alicyclic hydrocarbons, e.g. octane, nonane, decane and paraffins, including isomers thereof; petroleum fractions; mineral oil; kerosene; aromatic hydrocarbons, e.g. toluene, xylene, and naphthalene, including hydrogenated derivatives thereof, e.g. decalin and tetralin; halogenated hydrocarbons, e.g. monochlorobenzene; and cycloalkanes or cycloalkenes, e.g. careen, fluorine, camphene, menthane, dipentene, naphthalene, acenaphtalene, methylcyclopentandien, tricyclodecane, 1,2,4,5-tetramethyl-1,4-cyclohexadiene, fluorenone, naphtindane, tetramethyl-p-benzodiquinone, ethylfuorene, fluoranthene and naphthenone. Also combinations of the above-enumerated spinning solvents may be used for gel spinning of UHMWPE, the combination of solvents being also referred to for simplicity as spinning solvent. In one embodiment, the spinning solvent of choice has a low vapor pressure at room temperature, e.g. paraffin oil. It was also found that the process of the invention is especially advantageous for relatively volatile spinning solvents at room temperature, as for example decalin, tetralin and kerosene grades. Most preferably, the spinning solvent is decalin.

The combination of the large diameter and the low spin solvent residue makes the monofilament highly suitable for use in medical applications.

The diameter of 30 μm or more allows the monofilament to be used as a yarn without further twisting or fusing process, with an advantage that there is less possibility of bacteria harboring in pores.

The residual spin solvent content of 100 ppm or less makes the cumbersome cleaning process unnecessary for use in most medical applications. Preferably, the residual solvent content is 80 ppm or less and even more preferably, 60 ppm or less. The lower solvent content makes the monofilament yarn even more suitable for some special medical applications.

In one embodiment of the present invention, the yarn has a tenacity of 15 cN/dtex or more. Such tenacity is suitable for use in a mesh. In applications where especially high tenacity is required, such as a suture, the yarn preferably has a tenacity of 25 cN/dtex or more.

In one embodiment of the present invention, the surface roughness Ra of the monofilament is 300 nm or less, preferably 250 nm or less. The low surface roughness has an advantage that when used for medical applications such as a suture, a smooth stitching is obtained. The monofilament preferably has a low coefficient of variation of its linear density. The advantage is that it is more homogeneous, i.e. the monofilament shows less differentiation along its length in its mechanical and physical properties. The yarn has also improved mechanical and physical properties. Moreover, the yarn shows improved handling during manufacturing or use, especially at elevated speeds as for example in coating processes or in processes including yarn winding and/or high speed yarn transportation.

In another aspect of the present invention, a process for making a UHMwPE yarn is provided wherein the yarn is a monofilament. The process comprises the steps of:
a) spinning a solution filament from a solution of UHMwPE in a solvent;
b) cooling the solution filament obtained to form a gel filament;
c) drying the gel filament, during which the solvent is at least partly removed; and
d) drawing the gel filament in at least one drawing step after the step c) of drying the gel filament, to obtain a monofilament having a diameter of 30 μm or more and a residual spin solvent residue of less than 100 ppm, which monofilament forms the yarn.

It is a general prejudice in the art that filaments having a large diameter will have a problem in that removal of solvents by extraction or evaporation becomes increasingly difficult with increasing diameter. For example, EP0740002A1 states that thicker gel spun filaments would hinder the efficiency and completeness of the solvent removal. In EP0740002A1, this problem is solved by making a yarn with a higher diameter by fusing filaments of a smaller diameter. However, this process requires a step of fusing fibers in addition to the process of making the polyolefin filaments.

On the other hand, according to the present process, it was surprisingly found that the drying of the gel filament before drawing results in a residual solvent of less than 100 ppm in the final monofilament. It was also surprisingly found that drying the gel filament has no substantial affect on the drawability of the yarn.

The solution of UHMwPE in a solvent is spun from a spinplate comprising one spin hole or a plurality of spin holes. Preferably, the spinning of the filament is done in a manner in which the flow rate of the solution to be spun is controlled. In one embodiment, the solution of UHMwPE is spun from a spinplate comprising a flow rate control means present before the spin hole. The flow rate control means may be a metering pump. In an embodiment wherein the spin plate comprises a plurality of flow rate control means associated with different spin holes, each of the flow rate control means preferably controls the flow rate from the respective spin holes individually. Alternatively, the plurality of flow rate control means may also control the flow rate from different spin holes in the same manner.

The control of the solution flow rate is especially advantageous in this invention, since the effect of an inconstant flow rate is larger in making a larger diameter filament. A large diameter of the spin hole gives a higher possibility that the filament has a variation in its properties over its diameter. This will result in a more homogeneous monofilament.

The invention also relates to a medical device comprising the monofilament yarn according to the present invention. Such medical devices may be a surgical suture consisting of the yarn according to the present invention.

Surgical sutures must have an extremely high purity since it is used for stitching wound, which is susceptible to infection. A suture consisting of the yarn according to the present invention is especially advantageous because of its purity and less risk of attracting bacteria. Monofilaments have a stiff and smooth surface, which combine to reduce entanglement. This is also an advantage during the operation of closing wounds.

Another example is a surgical mesh comprising the monofilament yarn according to the present invention. The purity of the yarn and less risk of harboring bacteria are also advantageous for the surgical mesh. Moreover, the high flexibility and the light weight of the yarn makes it especially suitable for use as a mesh.

Another aspect of the present invention provides a use of the monofilament yarn according to the present invention for medical application. Good examples of products for medical application include sutures, mesh and cables, but also endless loop products, bag-like, balloon-like products and other woven and/or knitted products. Good examples of cables include a trauma fixation cable, a sternum closure cable, and a prophylactic or per prosthetic cable, long bone fracture fixation cable, small bone fracture fixation cable. Also tube-like products for e.g. ligament replacement are considered.

The invention will be explained more fully below with reference to the following example.

### Methods

IV: the Intrinsic Viscosity was determined according to method PTC-179 (Hercules Inc. Rev. Apr. 29, 1982) at 135° C. in decalin, the dissolution time being 16 hours, with DBPC as anti-oxidant in an amount of 2 g/l solution, by extrapolating the viscosity as measured at different concentrations to zero concentration.
Dtex: fibers' linear density (dtex, g/10 km) was measured by weighing a piece of fiber of predetermined length (about 18 cm). The dtex of the fiber was calculated by dividing the weight in milligrams by 10.
Tensile properties: tensile tests were carried out on an Instron Z010 tensile tester equipped with a 1 kN load cell and Instron parabolic fiber grips. Tensile strength was determined from the force at break and the linear density measured on each individual sample. Tensile modulus was determined as the chord modulus between 0.3 and 1.0% strain. Elongation at break and strain were determined by using a gauge length of 100 mm with a tension of 0.08 N at zero strain. The gauge length incorporated the full fiber length on the parabolic grip sections until the beginning of the flat pneumatical grip sections. Strain rate during tensile testing was 50 mm/min.
Diameter measurement: the diameter was calculated according to formula 1.
Solvent content: the solvent content was quantitatively measured via 2-step Multiple Headspace Extraction (MHE) gas chromatography (Agilent 6890N Gaschromatograph with an Alltech column filled with AT-wax and a flame ionization detector). A yarn specimen was put into a sealed headspace vial (Perkin Elmer headspace sampler Turbomatrix 40). The vial was placed into the desorption unit at a temperature of 135° C. for 30 minutes. The vapor phase was injected into the wax column and the solvent is detected with a flame ionization detector. The vial was depressurized and is heated up a second time to 135° C. for 30 minutes. The vapor phase was injected again and the recording of the chromatogram is repeated. The solvent content was calculated on the basis of the peak areas obtained for the sample in the two measurements and the calibration line.
Surface roughness: the surface roughness Ra was determined with an optical profilometer (Veeco NT1100). A yarn specimen was gold-coated before the measurement to prevent unwanted internal reflections. The yarn specimen was scanned using the VSI-mode (vertical scanning interferometry). After scanning, the profile was corrected by cylinder and tilt correction to adjust to the cylindrical shape of the filament using an operating-software of the optical profilometer. The surface roughness was calculated from the scanned data by the operating-software.

### EXAMPLE 1

A UHMwPE monofilament was made via a gel spinning process. A solution of 10.5 wt % of UHMwPE of IV 18 dl/g in decaline was spun at about 180° C. through a spin plate having one spin hole with a diameter of 2 mm into a solution monofilament. The solution monofilament was issued from the spin hole to an air-gap of 25 mm and entered a water bath. The solution monofilament was cooled in a water bath kept at about 20° C., and taken-up at such rate that a draw ratio of 3 was applied to the as-spun filaments in the air-gap. Spin velocity was kept constant at 0.64 m/min. The gel filament was dried in air at room temperature for 24 hours. The gel filaments were subsequently further drawn in two steps in an oil-heated tubular oven with a length of 90 cm. The first drawing step was carried out at 130° C. with a draw ratio of 5 by setting the entrance speed at 0.2 m/min and the exit speed at 1 m/min. The second drawing step was carried out at 149° C. with a draw ratio of 9.5 by setting the entrance speed at 0.063 m/min and the exit speed at 0.6 m/min.

The diameter, the residual solvent content and the surface roughness Ra of the yarn were measured according to the methods described hereinabove and the results are presented in Table 1. The yarn has a solvent content and a surface roughness Ra which are low enough to be used for medical applications.

TABLE 1

| | | Solvent | Surface |
|---|---|---|---|
| | Diameter | content | roughness Ra |
| | (μm) | (ppm) | (nm) |
| Ex. 1 | 55 | 94 | 224 |

The tensile properties of the yarn were also measured according to the methods described hereinabove and the results are presented in Table 2. The strength of the monofilament is also high enough to be used as a suture as well as a surgical mesh.

TABLE 2

| | Tenacity | E-modulus | Eab |
|---|---|---|---|
| | (cN/dTex) | (cN/dTex) | (%) |
| Ex. 1 | 28.5 | 1259 | 2.7 |

1. A UHMwPE yarn having a diameter of 30 μm or more and a residual spin solvent of less than 100 ppm, wherein the yarn is a gel-spun monofilament.

2. The UHMwPE yarn according to claim 1, wherein the yarn has a tenacity of 15 cN/dtex or more, preferably 25 cN/dtex or more.

3. The UHMwPE yarn according to claim 1, wherein the diameter is 40 μm or more, preferably 50 μm or more.

4. The UHMwPE yarn according to claim 1, wherein the residual spin solvent is less than 80 ppm, preferably less than 60 ppm.

5. The UHMwPE yarn according to claim 1 4, wherein the diameter is less than 150 μm, preferably less than 100 μm.

6. The UHMwPE yarn according to claim 1, wherein the yarn has a surface roughness Ra of less than 300 nm, preferably 250 nm or less.

7. A process for making a UHMwPE yarn, comprising the steps of:
a) spinning a solution filament from a solution of UHMwPE in a solvent;
b) cooling the solution filament obtained to form a gel filament;
c) drying the gel filament, during which the solvent is at least partly removed; and
d) drawing the gel filament in at least one drawing step after the step c) of drying the gel filament, to obtain a monofilament having a diameter of 30 μm or more and a residual spin solvent residue of less than 100 ppm, which monofilament forms the yarn.

8. The process according to claim 7, wherein the step c) of drying of the gel filament is performed at room temperature.

9. The process according to claim 7, wherein the solvent is decaline.

10. A suture consisting of the UHMwPE yarn according to claim 1.

11. The suture of claim 10, having USP 9-0, USP 8-0 or USP 7-0.

12. A cable consisting of the UHMwPE yarn according to claim 1.

13. A surgical mesh comprising the UHMwPE yarn according to claim 1.

14. A medical device comprising the UHMwPE yarn according to claim 1.

15. A use of the UHMwPE yarn according to claim 1 for medical application.
